(19) United States
(12) Patent Application Publication
(10) Pub. No.: US 2016/0051152 A1
(43) Pub. Date: Feb. 25, 2016

(54) METHOD FOR ESTIMATING BLOOD LOSS FROM FLUIDS COLLECTED DURING SURGERY

(71) Applicant: Anil Kumar, Rochester Hills, MI (US)

(72) Inventor: Anil Kumar, Rochester Hills, MI (US)

(21) Appl. No.: 14/832,255

(22) Filed: Aug. 21, 2015

Related U.S. Application Data
(60) Provisional application No. 62/040,612, filed on Aug. 22, 2014.

(51) Int. Cl. A61B 5/02; G01N 33/49; A61B 10/00

(52) U.S. Cl. CPC A61B 5/02042; A61B 10/0045; G01N 33/49

(57) ABSTRACT

The invention relates to a method for estimating blood loss from a sample of fluid collected during surgery. The method includes utilizing spectrophotometric analysis to determine a hemoglobin proportion within the sample, and estimating a patient's total blood loss based upon the analysis and known proportions of hemoglobin mass to blood volume.

### CROSS-REFERENCE TO RELATED APPLICATIONS

This application is a completion application of co-pending U.S. Provisional Patent Application, Ser. No. 62/040,612, filed Aug. 22, 2014 for “System and Method for Estimating Blood Loss from Fluids Collected During Surgery,” the entire disclosure of which is hereby incorporated by reference, including the drawings.

### BACKGROUND OF THE INVENTION

1. Field of the Invention

This disclosure relates generally to methods for analyzing a patient's fluid samples, and more particularly to methods for estimating blood loss by a patient during surgery.

2. Prior Art

Hemoglobin is the iron containing oxygen transport contained in red blood cells that functions to transport oxygen from the lungs to the rest of the body. Hemoglobin levels can be estimated using various known techniques including in vitro directly from a patient's fluid sample. Direct measurement of hemoglobin may be made using a blood sample from a patient in a testing setting rather than a clinical setting. Direct measurement of hemoglobin can be quite laborious and time intensive, requiring the lysing of red blood cells to free the hemoglobin into solution and then applying assay techniques to the resulting concentration of the hemoglobin in the solution. Direct measurement is additionally undesirable in time-critical medical situations when blood volume estimation and inferential blood-loss information must be readily obtainable. However, the act has recognized that there is a direct correlation between measured or estimated hemoglobin and blood volume.

Recently, optical-based systems have been developed to estimate hemoglobin quantities in a volume of a blood sample. One such type of optical-based system is a spectrophotometric system. In general, the term “spectrophotometric” refers to capturing spectral response over a range of wavelengths and correlating a response for each of the wavelengths. A device that performs this analysis is referred to as a “spectrophotometer,” an example of which includes a hemoglobinometer. Such spectrophotometric analysis has been performed with near-infrared and adjacent visible radiation, which is capable of ascertaining hemoglobin quantities in a fluid sample. The present invention seeks to capitalize on this to measure blood volumes.

In various types of surgical procedures and medical situations, a mixture of expelled bodily fluids are collected having unknown constituents with unknown proportions. Estimation of the expelled bodily fluid mixture constituents and associated volume can be highly medically important especially the blood volume. Measurement or estimation of the blood volume is particularly important during surgery and medical treatment situations when blood loss volume from the patient's bloodstream may be used to measure blood loss as well as when performing blood transfusions including calculating the flow rate into the patient's circulatory system. Therefore, it would be advantageous to estimate a volume of blood in a fluid sample that may be a mixture of various bodily fluid types to estimate blood loss by a patient.

### SUMMARY OF THE INVENTION

A method is described herein for estimating blood mass in a fluid sample of a collected bodily fluid volume. The fluid sample includes an unknown mixture of blood constituents and unknown bodily fluids that may be collected in various medical procedures, such as trauma-induced events and surgical-based settings, and has particular utility in urological procedures.

The method includes utilizing spectrophotometric analysis to determine a hemoglobin proportion within a sample of a bodily fluid collected during a surgical procedure, and estimating blood quantity based upon the analysis and known proportions of hemoglobin mass to blood volume.

In accordance herewith the estimation of the amount of blood loss during surgery comprises: (a) collecting a volume of a fluid sample during the surgery and (b) estimating the amount of volume of blood loss using a spectrophotometric analysis.

This is done by determining a hemoglobin proportion within the collected sample and therefrom estimating the volume of blood loss in accordance with the following equation:

$$\frac{Y ( volume collected ) \times X ( amount of hemoglobin per deciliter )}{14 gms \text{/} dcl}$$

wherein Y (volume of the sample collected) multiplied by X (the amount of hemoglobin per deciliter) divided by the known average of fourteen grams of hemoglobin per deciliter.

The amount of hemoglobin per deciliter is measured with a spectrophotometer or hemoglobinometer.

Alternatively, the patient's actual percentage of hemoglobin per deciliter can be used in lieu thereof.

The spectrophotometer or hemoglobinometer is in communication with, preferably, a computer, which provides substantially instantaneous calculation.

Once the calculation is achieved, it is possible to thereby estimate the amount of blood loss in the total fluid collected.

Various embodiments of the present invention will be described in detail with reference to the drawings, where like reference numerals represent like parts and assemblies throughout the several views, in which reference to various embodiments does not limit the scope of the invention, which is limited only by the scope of the claims attached hereto.

### BRIEF DESCRIPTION OF THE DRAWINGS

One or more embodiments will now be described, by way of example, with reference to the accompanying drawings, in which:

FIG. 1 schematically shows an exemplary medical system, in accordance with the present disclosure;

FIG. 2 shows a flow chart of the process associated with operation of the medical system, in accordance with the present disclosure; and

FIG. 3 shows an exemplary computing device of the system, in accordance with the present disclosure.

### DESCRIPTION OF THE INVENTION

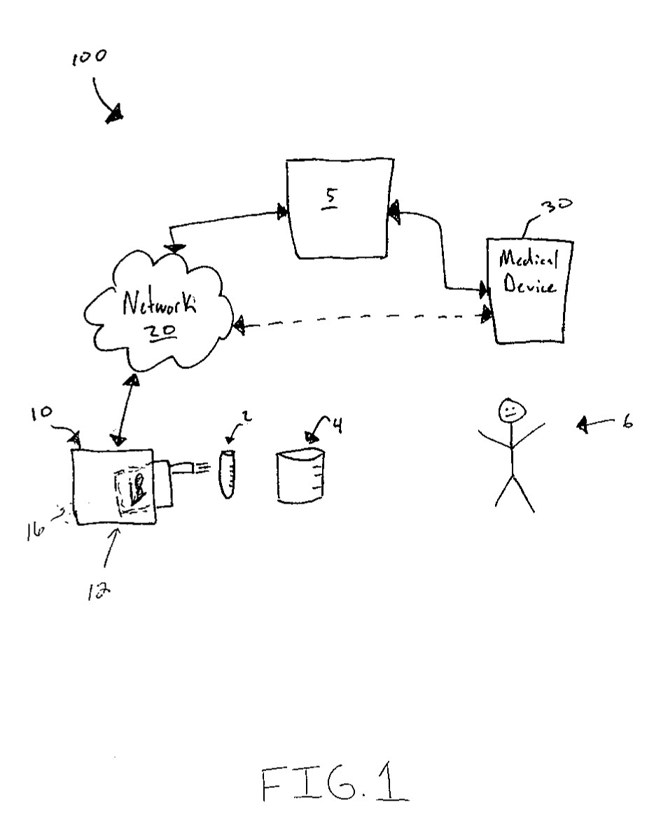

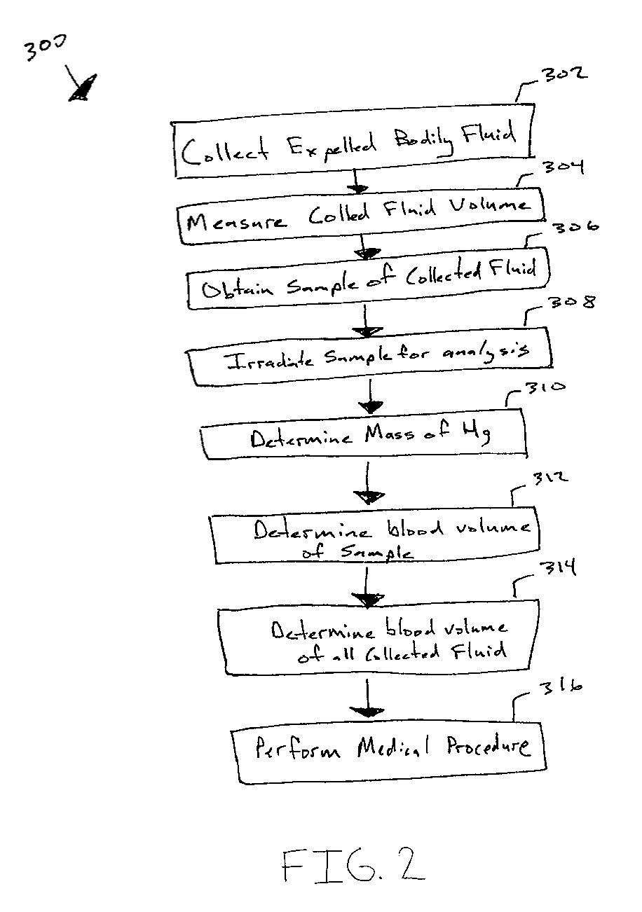

Referring to FIGS. 1 and 2, there is depicted a flow chart 300 for a process 20 associated with operation of a procedure 100 for estimating blood mass or volume in collected serological fluid during or after surgery. As illustrated, this process is initiated at step 302 by a medical professional or other operator-user of the system 100 by collecting the total serological fluid 4 from a patient 6 during surgery. At step 304, a volume measurement of the fluid is obtained. At step 306, a sample 2 is collected having a known volume from fluid 4.

At step 308, the sample is analyzed using any suitable fluid analyzer 10, including a spectrophotometer, a hemoglobinometer, or the like. Hemoglobinometers are well-known and commercially available. Such fluid analyzers perform spectrophotometric analysis of the sample 2 using irradiated optical energy at near infrared or infrared wavelengths. A spectrophotometric analysis determines a proportion of hemoglobin within the sample 20, e.g., 1.00%.

At step 310, a mass of hemoglobin is determined based upon the proportion identified within the sample 2 and known hemoglobin properties. At step 312 a volume of blood constituent within the sample 2 is determined based upon the determined hemoglobin mass and a known proportion of hemoglobin to unit volume of blood fluid, e.g., 14 grams hemoglobin to 100-milliliters of blood fluid. Lastly, the volume of blood within the fluid 4 is extrapolated from the determination of the blood volume determined within the sample 2 at step 314. Step 308 through step 314 may be expressed as:

$$\frac{14 g * V ( sample ) * V ( mixture )}{100 ml * H ( sample )} = BV ( mixture )$$

wherein V(sample) is the volume of the sample size, V(mixture) is the volume of the collected mixture size, BV(mixture) is the blood volume of the mixture, and H(sample) is the hemoglobin mass determined using spectrophotometric analysis. Alternatively, this may be done by determining a hemoglobin proportion within the collected sample and therefrom estimating the volume of blood loss in the total fluid collected in accordance with the following equation:

$$\frac{Y ( volume collected ) \times X ( amount of hemoglobin per deciliter )}{14 gms \text{/} dcl}$$

wherein Y (volume of the sample collected) multiplied by X (the amount of hemoglobin per deciliter) divided by the known average of fourteen grams of hemoglobin per deciliter.

At step 314 the medical device 30 utilizes the determined blood volume in the sample 2 to estimate the total blood volume of the collected fluid.

Estimation of the blood volume is used to infer blood loss within the total fluid collected by the patient. Therefrom, it is possible to determine the need for a transfusion or other treatment at step 316. It is contemplated by the disclosure herein that the blood volume of the mixture may be iteratively estimated, whereby rolling averages may be calculated enabling extrapolation and forecasting of potential blood loss.

Referring again to the drawing, FIG. 1 schematically shows an exemplary medical system 100 for estimating blood loss in the collected fluid in accordance with the present invention. The system 100 includes a computing device 5, and a fluid analyzer 10. The computing device 5 is communicatively connected to the fluid analyzer 10. The fluid analyzer 10 may be any suitable spectrophotometer or hemoglobinometer device. Components of the system 100 are shown in FIG. 1 as discrete elements. Such illustration is for ease of description and it should be recognized that the illustrated components may be implemented in any number of separate components including as a single device and may include additional computing and medical devices, e.g., a computer server.

The computing device 5 may be of any one of various types of computers including microcomputers, minicomputers, mainframes, and/or data storage devices. The computing device 5 preferably executes database functions including storing and maintaining a database and processes requests from the fluid analyzer 10 to extract data from, or update, a database as described below herein. The computing device 5 may additionally provide processing functions for the fluid analyzer 10.

The fluid analyzer 10 is any suitable spectrophotometer or hemoglobinometer that operates acceptably and is, preferably, responsive to radiation in the near-infrared and adjacent visible light regions with sufficient precision. The term “near-infrared and adjacent visible radiation” or light herein refers to radiation between about 400 and 2500 nm, and preferably between about 475 and 1075 nm. The fluid analyzer 10 includes a main operational unit 12 and an operator control unit 14. Preferably, the operator control unit may be implemented on the computing device 5.

The operational unit 12 preferably, includes an outer housing 16 and a sample processing portion 18 for conducting the desired clinical tests or measurements of a sample 2.

The fluid analyzer 10 may include one or more applications that the user may operate. Operation may include downloading, installing, turning on, unlocking, activating, or otherwise using the application. The application may comprise at least one algorithm, software, computer code, and/or the like, for example, mobile application software. In the alternative, the application may be accessible via a website or web-based computer page accessible through the network 20 and controlled via the computing device 5.

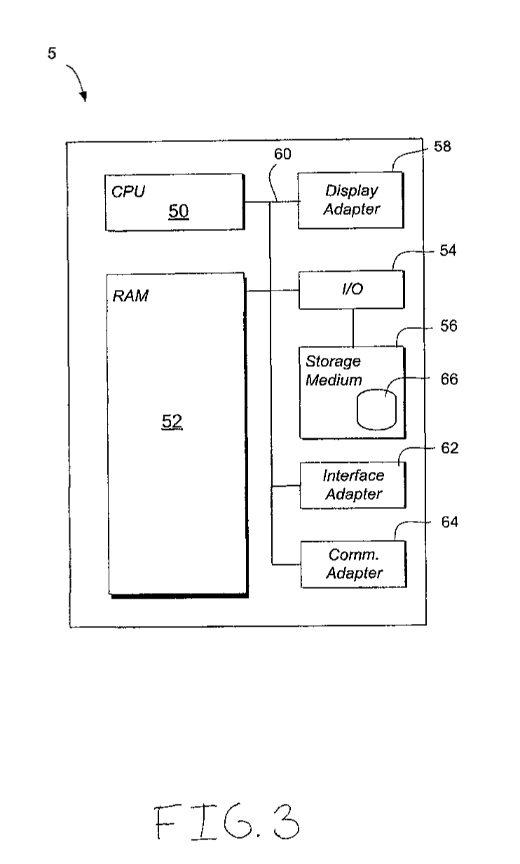

FIG. 3 shows an exemplary computing device 5. The computing device 5 includes a central processing unit (CPU) 50, random access memory (RAM) 52, input/output circuitry 54 for connecting peripheral devices such as a storage medium 56 to a system bus 60, a display adapter 58 for connecting the system bus 60 to a display device, a user interface adapter 62 for connecting user input devices such as a keyboard, a mouse, and/or a microphone, to the system bus 60. The device 5 also includes a wireless adapter configured for extraterrestrial communication such as in a communications satellite. The storage medium 56 is configured to store, access, and modify a database 66, and is, preferably, configured to store, access, and modify structured or unstructured databases for data including, for example, relational data, tabular data, audio/video data, and graphical data.

The central processing unit 50 is preferably one or more general-purpose microprocessor or central processing unit(s) and has a set of control algorithms, comprising resident program instructions and calibrations stored in the memory 52 and executed to provide the desired functions including parallel processing functions. As one skilled in the art will recognize, the central processing unit 50 may have any number of processing “cores” or electronic architecture configured to execute processes in parallel.

An application program interface (API) is preferably executed by the operating system for computer applications to make requests of the operating system or other computer applications. Of course platforms and operating systems other than those described herein may be used with equal efficacy.

In an alternate embodiment hereof, the system 100 may include a network 200 and a medical device 30 for monitoring or performing procedures on a patient 6.

Here, the medical device 30 is interposed the network 200 or is directly connected to the fluid analyzer 10 in one implementation.

The device may be physically connected and disconnected to the network 200 or the computing device 5 during selected periods of operation without departing from the teachings herein.

The network 200 may be any suitable series of points or nodes interconnected by communication paths. The network 20 may be interconnected with other networks and contain sub networks network such as, for example, a publicly accessible distributed network like the Internet or other telecommunications networks (e.g., intranets, virtual nets, overlay networks and the like) and may include a communication adapter 64 for connecting the computing device 5 to the network 200.

Where used, network 200 may facilitate the exchange of data between and among the fluid analyzer 10, the computing device 5, and the medical device 30.

It is to be appreciated that the present invention provides a means and method for a surgeon to estimate blood loss during surgery to assist in safeguarding the patient and better determine the next course of action in light of the amount of blood loss.

1. A method for calculating the estimated blood loss of a patient during a surgical procedure comprising:
(a) collecting expelled bodily fluid;
(b) obtaining a sample of the fluid;
(c) determining the volume of the sample;
(d) determining the proportion and the mass of the amount of hemoglobin within the sample;
(e) calculating the volume of blood in the sample; and
(f) extrapolating the volume of blood in the sample to determine the amount of blood in the fluid using one of the following equations:
$$\frac{14 g * V ( sample ) * V ( mixture )}{100 ml * H ( sample )} = BV ( mixture )$$
wherein V(sample) is the volume of the sample, V(mixture) is the volume of the fluid, BV(mixture) is the volume of blood the fluid, and H(sample) is the mass of the hemoglobin; or:
$$\frac{Y ( volume collected ) \times X ( amount of hemoglobin per deciliter )}{14 gms \text{/} dcl}$$
wherein Y is the volume of the sample, multiplied by X, which is the mass of hemoglobin, and divided by a known average of fourteen grams of hemoglobin per deciliter.

2. The method of claim 1 wherein the fluid is analyzed using a fluid analyzer.

3. The method of claim 2 wherein the fluid analyzer is a spectrophotometer.

4. The method of claim 2 wherein the fluid analyzer is a hemoglobinometer.

5. The method of claim 2 wherein the fluid analyzer is communicatively connected to a computing device; the computing device further comprising:
(a) a central processing unit; a random access memory;
(b) an input/output circuitry for connecting a plurality of peripheral devices such as a storage medium to a system bus;
(c) a display adapter for connecting the system bys to a display device;
(d) a user interface adapter for connecting user input devices such as a keyboard, a mouse, and/or a microphone to the system bus; and
(e) a wireless adapter configured for extraterrestrial communications.

6. The method of claim 5 wherein the computing device is a microcomputer, minicomputer, mainframe, data storage device, or the like; the computing device further comprises:
(a) the ability to provide processing functions for the fluid analyzer; and
(b) the ability to execute database functions including storing and maintaining a database and processes requests from the fluid analyzer to extract data from, or update, the database.

7. The method of claim 2 wherein the fluid analyzer is responsive to radiation in the near-infrared and adjacent visible light regions between about 400 and 2500 nm with sufficient precision.

8. The method of claim 2 wherein the fluid analyzer further comprises an operational unit and an operator control unit; the operational unit further comprising an outer housing and a sample processing portion.

9. The method of claim 2 wherein the fluid analyzer is directly connected to a network and a medical device for monitoring or performing procedures on a patient.
